(19) United States
(12) Patent Application Publication
(10) Pub. No.: US 2021/0260465 A1
(43) Pub. Date: Aug. 26, 2021

(54) VISUALIZATION AND ANALYSIS OF THERAPEUTIC REGIMEN EFFECTS ON HUMAN PHYSICAL PERFORMANCE METRICS

(71) Applicant: iParametrics, LLC, Alpharetta, GA (US)

(72) Inventors: Andrew J. Klein, Alpharetta, GA (US); John M. Lorimer, Alpharetta, GA (US); Paul S. Pelletier, JR., Alpharetta, GA (US)

(21) Appl. No.: 17/181,818

(22) Filed: Feb. 22, 2021

Related U.S. Application Data
(60) Provisional application No. 62/979,609, filed on Feb. 21, 2020.

(51) Int. Cl. A63B 71/06; G06F 3/0481; G06F 3/0484; G06N 20/00; G06N 5/04; A63B 24/00

(52) U.S. Cl. CPC A63B 71/0622; G06F 3/0481; G06F 3/04842; A63B 2071/0675; G06N 5/04; A63B 24/0075; G06N 20/00

(57) ABSTRACT

Disclosed are various embodiments for the visualization and analysis of therapeutic regimen effects on human physical performance metrics. In one embodiment, data is obtained respecting at least one population of individuals having a respective physical impairment. The data indicates for each of the individuals a respective subset of a set of therapeutic regimens used to treat the respective physical impairment, and a set of performance measures of the respective individual following the respective subset of the set of therapeutic regimens. The data is analyzed to automatically determine a reduced set of therapeutic regimens and a reduced set of performance measures. A user interface is generated that facilitates a comparison of individual ones of the reduced set of therapeutic regimens to the reduced set of performance measures for the at least one population.

### CROSS-REFERENCE TO RELATED APPLICATIONS

This application claims the benefit of U.S. Provisional Application 62/979,609, entitled “VISUALIZATION AND ANALYSIS OF THERAPEUTIC REGIMEN EFFECTS ON HUMAN PHYSICAL PERFORMANCE METRICS,” and filed on Feb. 21, 2020, which is incorporated herein by reference in its entirety.

### BACKGROUND

In many occupations, physical performance of tasks is necessary. Particularly for military service, emergency response, and athletics, workers may need to meet various physical performance metrics in order to perform their jobs effectively. With such workers, much of their training may focus on physical performance readiness for extreme situations. Failure to meet minimum physical performance criteria may result in disastrous consequences on the battlefield or when responding to an emergency.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 shows an example user interface for visualization of the relationship of performance and conditioning to impairments.

FIG. 2 shows an example user interface for visualization of the effect of therapeutic regimens on performance and injury/disability.

FIG. 3 shows an example user interface involving a population comparison dashboard.

FIG. 4 shows an example of a population comparison dashboard with continuous percentiles.

FIG. 5 shows an example of a population comparison dashboard with fractional standard deviation buckets.

FIG. 6 shows an example user interface that shows a first population that appears to be doing better than a second population on all performance measures.

FIG. 7 shows an example user interface with an injury/disability filter selected.

FIG. 8 shows an example user interface with an interactive mode selected.

FIG. 9 shows an example user interface with an interactive mode selected and the second population selected.

FIG. 10 shows an example user interface with the interactive mode selected, the second population selected, and the shoulder impairment selected.

FIG. 11 shows an example user interface with interactive mode selected, cleared populations, and impairment filters.

FIG. 12 shows an example user interface in interactive mode with the “None” option for the therapeutic regimen of “physical therapy.”

FIG. 13 is a schematic block diagram of a networked environment according to various embodiments of the present disclosure.

FIG. 14 is a flowchart illustrating one example of functionality implemented as portions of an enhancement application executed in a computing environment in the networked environment of FIG. 13 according to various embodiments of the present disclosure.

FIG. 15 is a schematic block diagram that provides one example illustration of a computing environment employed in the networked environment of FIG. 13 according to various embodiments of the present disclosure.

### DETAILED DESCRIPTION

The present disclosure relates to visualization and interactive analysis of the effects of various types of therapeutic regimens on human physical performance metrics. It is important to optimize a training or conditioning program so that individuals are able to perform at their best. However, it may be difficult to ascertain which components of a training or conditioning program will having meaningful impacts on key performance measures.

Various embodiments of the present disclosure introduce approaches to visualize the effects of various types of therapeutic regimens on human performance metrics. Therapeutic regimens are various treatments that are imposed over time, like medical treatments, physical therapy, conditioning regimens, psychiatric treatment, and so on. Performance measures are the results over time of various repeated physical activities like time for a five-mile run, amount of weight lifted in a bench press, and so on that represent an individual's state of physical fitness. The performance measures themselves can be a type of therapeutic regimen in that they bring about improvement, but they are also used as a proxy to measure improvement. That is, performance measures are both an input and output of the process.

Large volumes of data may be available relating to various metrics of performance and therapeutic regimens. Machine learning as applied to this data may be used to allow user to explore, evaluate, and demonstrate the impact of various therapeutic regimens on improvement of physical performance. Non-limiting examples of therapeutic regimens may include physical therapy, medicine, nutrition, and mental health. A user interface may allow the user to visually compare two or more populations and analyze and optimize across multiple complex variables.

In one example, there are three interactive variable categories with multiple variables within each. It may be noted that as the number of variables and number of types (categories/dimensions) of variables increases, it becomes increasing difficult for a person to manually assess the complex interaction between them. This system provides a methodology for optimizing these variables against a desired outcome through a combination of machine learning and visualization.

In various embodiments, an enhancement application allows the user to filter the data based on various measures and dimensions. For example, users can filter impairment types by region, type and nature. Also, users can filter the reference population and comparison populations by organization, position, gender and age. The enhancement application allows the user to perform comparisons of two different populations based on various dimensions (e.g., compare Army to Navy). Comparisons can be made, for example, on a percentage basis or on the basis of standard deviation buckets.

In one implementation, the enhancement application allows the user to display and manipulate four different performance measures against four different therapeutic regimens and multiple types of Impairments. The four performance measures included are those that had been determined through machine learning techniques to be the ones most correlated with changes in the therapeutic regimens. Different quantities may be used in different implementations.

The enhancement application may offer both a historical mode and an interactive mode. The historical mode may display results as they exist in the historical data. The interactive mode may allow the user to set either certain performance measures or therapeutic regimens or both to certain target values and the system will display the relative impact of these changes based on the historic data. In other words, this allows the user to conduct various “what if” scenarios, along the lines of: “If I maximize the Nutrition therapeutic regimen, how would this impact the Five Mile Run statistics?” In this way, the user may select potentially optimal therapeutic regimens to focus on, while minimizing potentially less effective ones.

In one example, data may be available with several hundred different performance measures and many therapeutic regimen types. One goal of the project may be to identify those therapeutic regimens that are most correlated with changes in performance measures. Note that this is a “multivariate” analysis because it involves finding the best combinations of performance measures related to the best combinations of therapeutic regimens and with respect to potential injuries/disabilities over time. Machine learning methods may be used to “feature engineer” the best combinations, resulting in a selection of a limited number of performance measures with respect to a limited number of therapeutic regimens with respect to injury/disability and time that were most optimal, thereby reducing the complexity of the visual analysis. In one example, the number of performance measures may be four, but other numbers may be used in other examples. Among other things, the user interface is designed to prevent the user from running scenarios that would lead to invalid or misleading results.

In other embodiments, a machine learning approach is used to generate predictive models that accomplish the goals of the user interface directly, without user intervention. Predictive modeling techniques are extremely efficient at analyzing extremely complex multi-variate relationships. Machine learning models may optimize answers to various questions across all of the variables discussed here and directly generate an output. For example, rather than the user using a user interface to try to determine manually the answer to the following question, a model may generate the answer automatically and pro-actively: “What is the best set of therapeutic regimens that will accomplish the greatest improvement in performance for someone from the Navy branch of military service who has experienced a knee injury?” In this way, the system becomes prescriptive.

In perhaps an even more general sense, the machine learning algorithms and the user interface allows users to dynamically compare two different populations across multiple dimensions as a means of determining optimal combinations of factors that suggest means to optimize results. In this current instance, the multiple factors are certain specific performance measures, certain specific injury types, certain specific therapeutic regimens across time and changes over time, with a goal of determining which therapeutic regimens have the greatest positive impact on improvements in human performance across multiple dimensions.

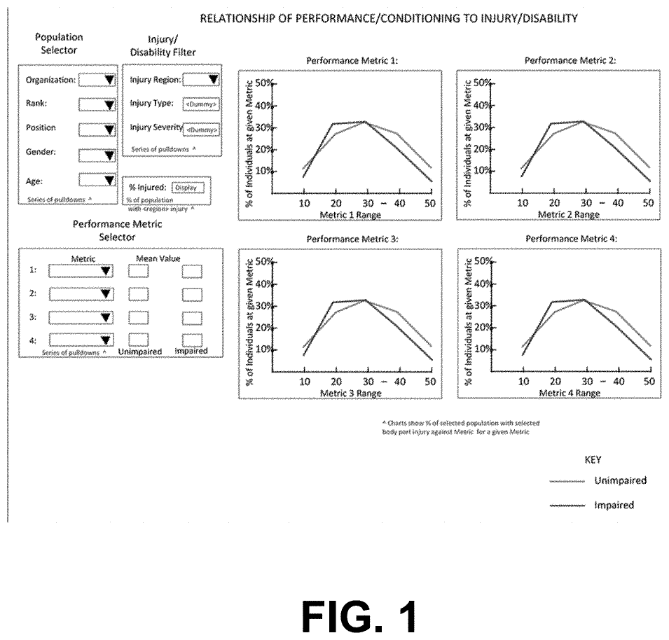

FIG. 1 shows an example user interface for visualization of the relationship of performance and conditioning to impairments. A purpose of this user interface is to illustrate how individual performance measures reflect a propensity toward injury or impairment. For example, does a higher value for a given performance measure seem to be related to less knee injury? That is, if the curve for the injured population is significantly to the left of, or worse than, the curve for the uninjured population, then the implication may be “yes”. Multiple performance measures may be compared at a time. This chart may represent a single point in time. The impaired performance measure may be measured as close to the date of injury or impairment onset as possible. Because many factors may be related to impairment, these results may be considered directional instead of definitive.

The variables used in FIG. 1 may include a performance measure metric, a type of disability or injury, and impaired and unimpaired populations. The performance measure may be a continuous scale of a low to a high bound of the metric, or a continuous scale of a low to a high bound of a performance index score. However, not all performance measures may have an index score. The type of injury/disability may include discrete filters based on different body regions. The impaired and unimpaired populations may include discrete filters. Being impaired may be defined as having a selected type of impairment at any point during a measurement period. The interface may be configured not to allow selection of a single individual. The number of performance measure options may be limited to a number such as eight, or another number. The selection may be assisted by machine learning analysis of which is most relevant.

The X axis for each of the charts may represent the performance measure “score” (Index value range or actual performance measure value range) for the selected performance measure. The Y axis for each of the charts may represent the number of individuals at a given performance measure metric.

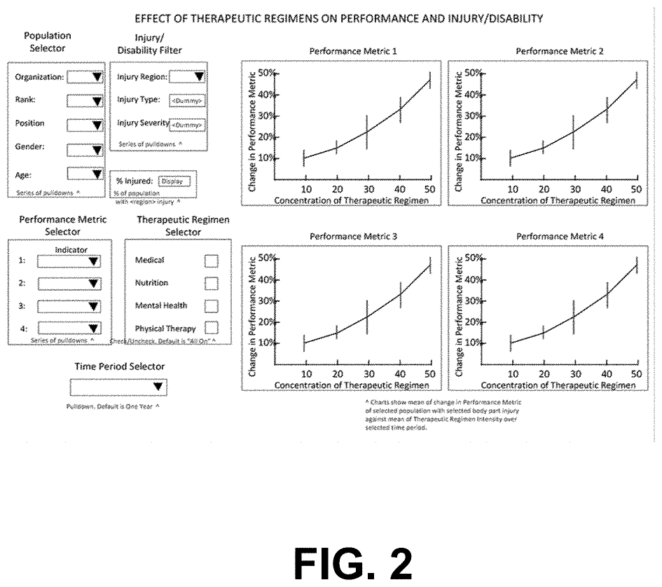

FIG. 2 shows an example user interface for visualization of the effect of therapeutic regimens on performance and injury/disability. A purpose of this user interface is to answer the questions: Do therapeutic regimens (therapeutic regimens) applied demonstrably impact performance improvement? If so, by how much? If so, which therapeutic regimens impact which performance metrics most? therapeutic regimens applied demonstrably impact recovery from injury/disability (impairment)? If so, by how much? If so, which therapeutic regimens impact recovery most? For example, does a higher therapeutic regimen concentration applied over one year result in a greater change in performance score during the same time period? If so, this implies that higher therapeutic regimen concentrations result in impairment recovery improvement. Also, specifically which therapeutic regimen types seem to result in the greatest levels of improvement? This user interface may take into account duration of therapeutic regimen. Even though this user interface answers multiple questions, this user interface may include one chart, since the performance measures are one measurement of “recovery” available. That is to say, a positive change in certain performance measures after an injury or disability is taken to be an indication of impairment recovery.

The variables in use for FIG. 2 include concentration of therapeutic regimen, changes in given performance measure values over time, time periods, types of impairment, and impaired and unimpaired populations. “therapeutic regimen Intensity” may be defined as sum of all therapeutic regimens (frequency)×therapeutic regimen duration for a given individual. This may also include therapeutic regimen “intensity” (e.g. frequency×duration×intensity). Filters may include Total population/Uninjured population/injured population, Population sub-segments (Organization, Rank, etc.), Type of Injury/Disability, Encounter Types, and Measurement Time Frame.

The user interface of FIG. 2 may take into account therapeutic regimens of medical, nutrition, mental health, physical therapy, and/or other therapeutic regimens. The user interface will allow for any one of the types of variables to be turned “on” or “off”, individually or in combination. Turning a therapeutic regimens type “off” may suppress it from the calculations and display. The X axis may depict the mean of total Concentration of therapeutic regimen over the specified time period in “buckets”. The Y axis may depict the amount of change in a given performance measure over the specified time period. The therapeutic regimen selector may allow for certain therapeutic regimen types to be included (“turned on”) or excluded (“turned off”) for the calculation. The default may be “all on”.

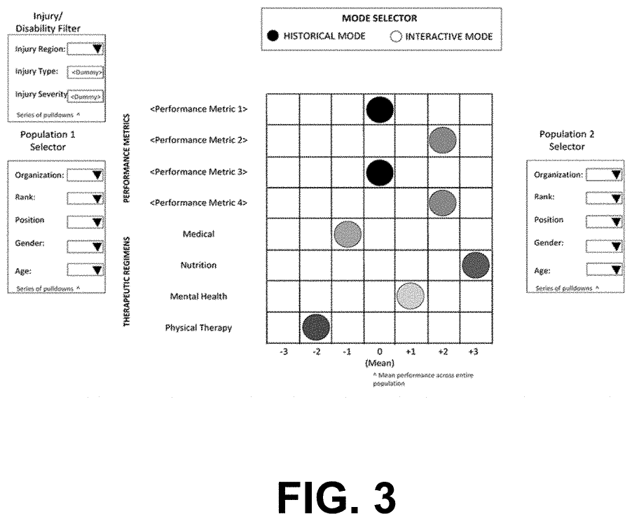

FIG. 3 shows an example user interface involving a population comparison dashboard. A purpose of this user interface is to answer the questions: In what ways does one given selected population compare to a normal population, or to another, different selected population? Where should a focus be on training/therapeutic regimen to improve an underperforming population? This can be answered with respect to performance measures or therapeutic regimen concentration. Also, the user interface may answer what happens if a given performance measure or therapeutic regimen intensity is improved. For example, this user interface may assist in determining are the Marines in better shape than the Army? If so, in what ways? How does an impaired population differ from an unimpaired population? Specifically, which performance measures and which therapeutic regiment types show the most difference? What kinds of therapeutic regimens might be most likely to be successful in treating a knee injury? In one implementation, the user interface may represent a current state of the data and may not take into account duration of therapeutic regimen.

Various features may be present in embodiments. A selection option limitation feature provides Hierarchical Selector options that are dynamically driven. That is, selecting a specific “Organization” causes the subsequent selectors to only display values appropriate to the selected organization. For example, some Ranks may not be appropriate to a given organization; in which case only relevant Ranks would be selectable.

A minimum population limitation feature may be present. For any given user interface and set of selectors, there is a minimum size of population that generates meaningful results. If the population resulting from a given set of selections (filters) is too small to be meaningful, either option will result in a “Not Applicable” warning and the data will not be displayed, or the data will be displayed but with a warning that the results are not relevant.

An injury risk score is a dynamically calculated, multi-variate, machine-learning based “Risk Score” denoting potential risk of injury based on the status of performance measures. An therapeutic regimen effectiveness score is a dynamically calculated, multi-variate, machine-learning based “Risk Score” denoting the relationship between performance measures and therapeutic regimens (duration and frequency) with prevention of injury and effectiveness of recovery from injury.

Variables may include mean and deviation from mean for performance measure metrics, mean and deviation from mean for therapeutic regimen type metrics, type of injury or disability, and impaired and unimpaired populations. Filters may include total population, uninjured population, and injured population; population sub-segments (Organization, Rank, etc.), type of impairment, and a target value for performance measures and therapeutic regimen type.

The Horizontal (X) scale may represent mean of a first population and deviations from that mean from −3 to +3 for a second population. Scale is relative only and may not represent standard deviations from mean. The Y Axis represents this data for each of a number of individual performance measures and for four therapeutic regimen types, in one implementation.

If a given performance measure/delta combination is selected, the user interface may dynamically filter the reference population to select only those individuals that fall within that performance measure delta from mean and then display the comparison delta for the entire population for the other performance measures.

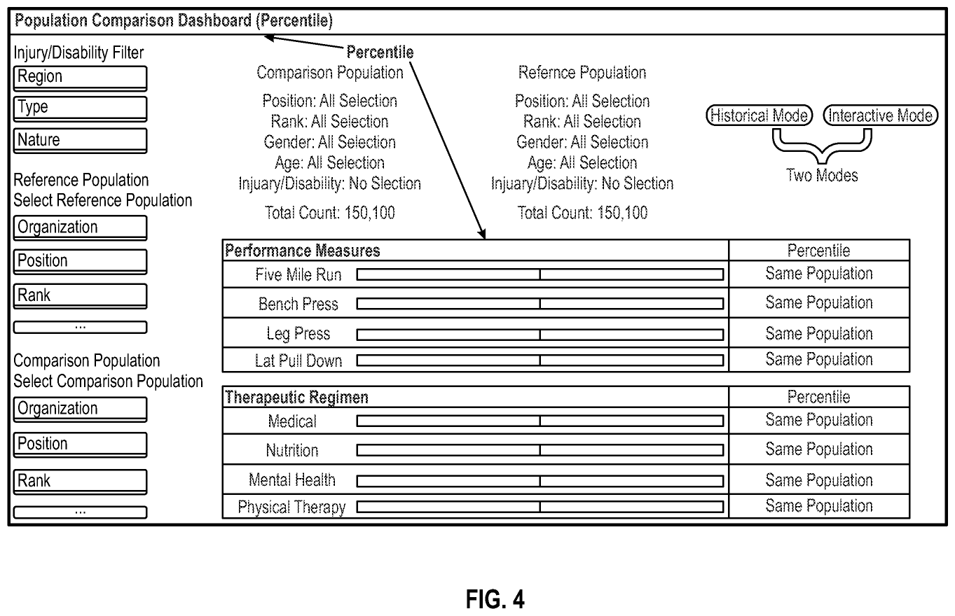

FIG. 4 shows an example of a population comparison dashboard with continuous percentiles.

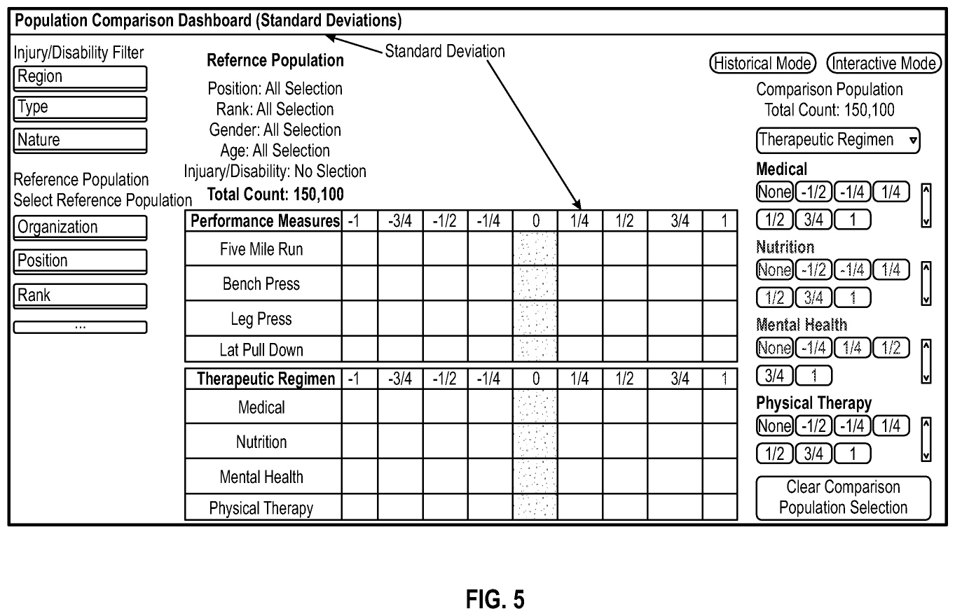

FIG. 5 shows an example of a population comparison dashboard with fractional standard deviation buckets.

These two examples versions in FIGS. 4 & 5 show the same data with somewhat different emphases. The Fractional Standard Deviation Buckets version visually illustrates the concept of “better or worse.” That is, green boxes to the right is “better,” red boxes to the left is “worse.” The Continuous Percentiles version visually illustrates the concept of “how much better or worse.” That is, the farther the bar is to the right, the better the metric. The initial state of the interfaces shows the “neutral” state: no filters selected, all parameters showing all individuals at the median (“0” standard deviation from median).

Two populations are used: one population is being compared to another population. Selectors allow these populations to be manipulated. Two categories of parameters are being measured: performance measures and therapeutic regimens. The interface shows the relationships between them. In addition, there is a filter for type of injury/disability. The multiple dimensions being represented can be managed—performance measures, therapeutic regimens, injury/disability, and time.

Indicia representing different populations can be used to select the populations. Counts of different populations can be shown. In one example, positive values are green, negative values are red with the amount of shading related to magnitude.

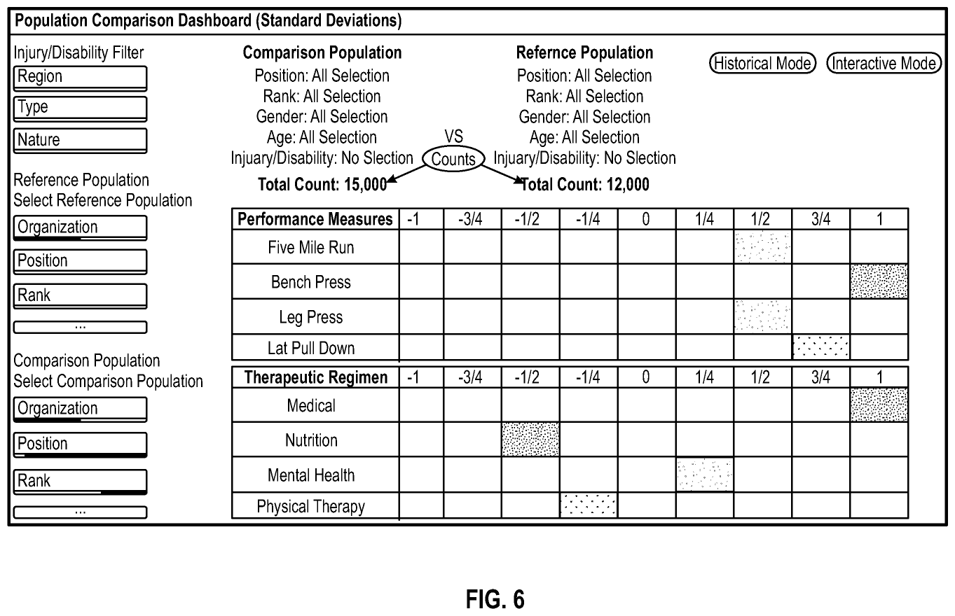

FIG. 6 shows an example user interface that shows a first population that appears to be doing better than a second population on all performance measures. On therapeutic regiments, the first population is weaker in taking advantage of nutrition and physical therapy.

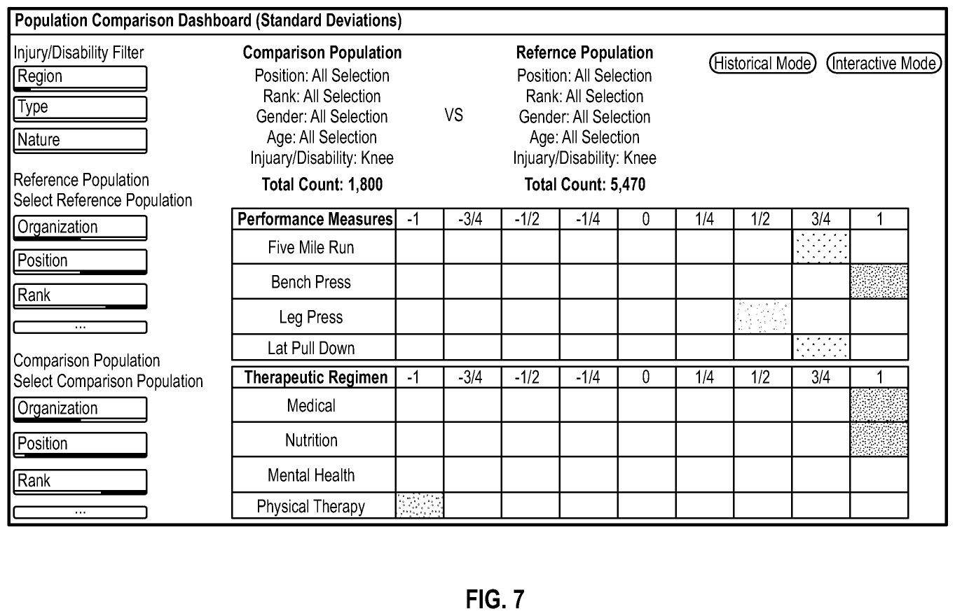

FIG. 7 shows an example user interface with an injury/disability filter selected (“Knee Injury”). The performance measures are similar (five mile run changes a bit), but this shows a great increase in nutrition and drop in physical therapy. It is possible, for example, that the second population is utilizing aggressive physical therapy post injury and the first population is not.

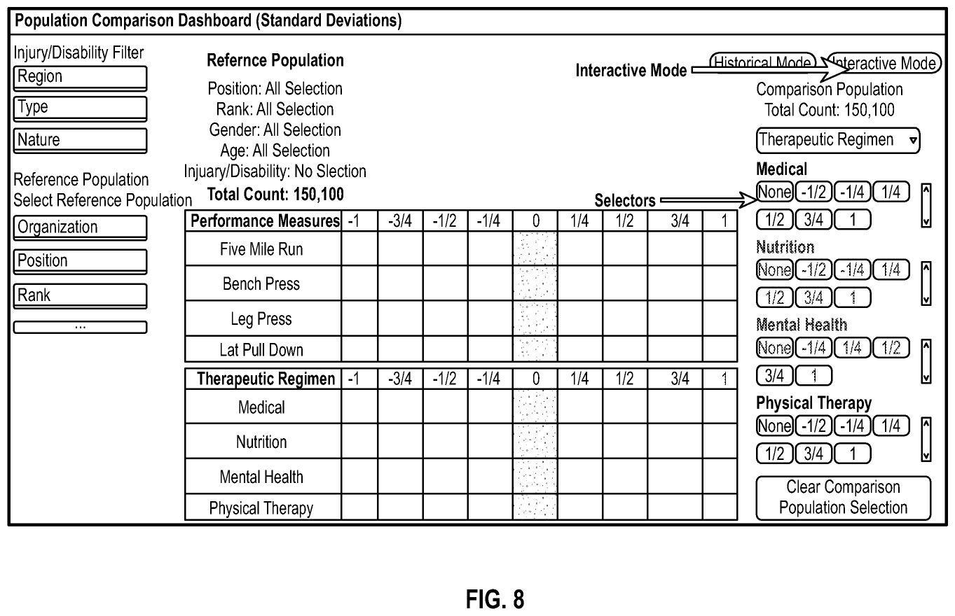

FIG. 8 shows an example user interface with an interactive mode selected. The interactive mode allows the user to perform various “what if” scenarios. When one (or more) of the parameters is selected (e.g. “−½”) this will divide the data into two populations. One population (the “comparison” population) will be only those individuals that fall one-half of a standard deviation or more below the median. The “reference” population will then be “everyone else,” i.e., the total selected population without those who fall one half of a standard deviation or more below the median.

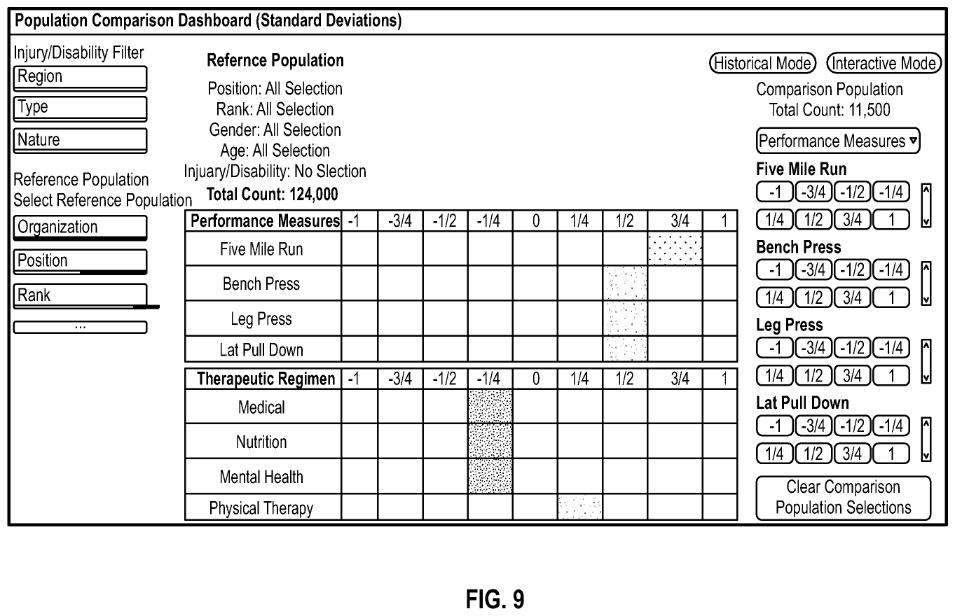

FIG. 9 shows an example user interface with an interactive mode selected and the second population selected. “+¾” is selected under “Five Mile Run.” Note that the other performance measures are also now high. This may imply, for example, that if an individual does really well on the five-mile run that their general conditioning is also likely to be good, as reflected in the other performance measures. Note also that their scores for physical therapy are also high, which may imply that a reason for their good performance is aggressive physical therapy.

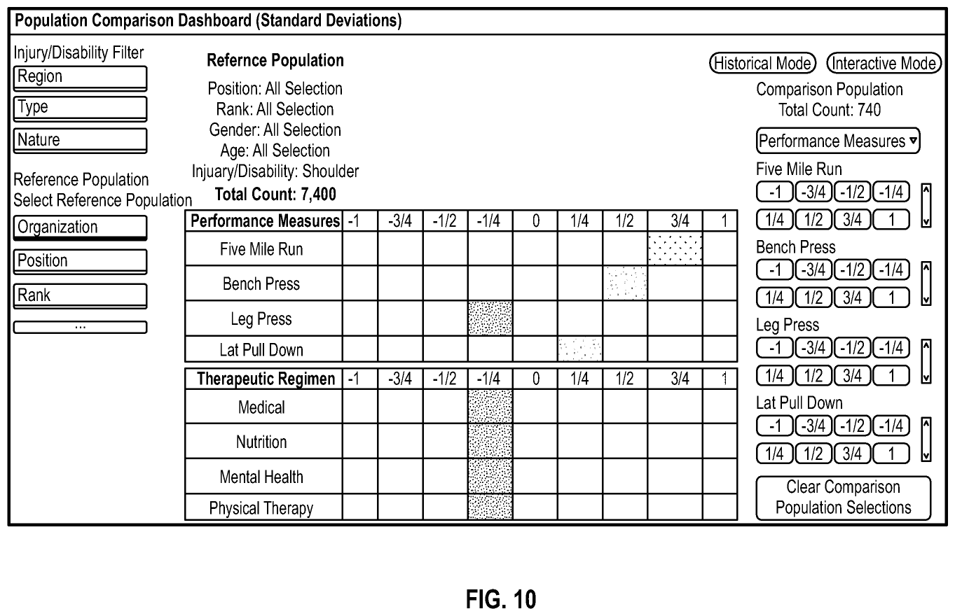

FIG. 10 shows an example user interface with the interactive mode selected, the second population selected, and the shoulder impairment selected. Note that the leg press and lat pull down metrics fall, perhaps showing the general reduction in overall performance due to the injury. Also note drop in physical therapy. This may imply that the individuals are not able to engage in physical therapy to the same extent.

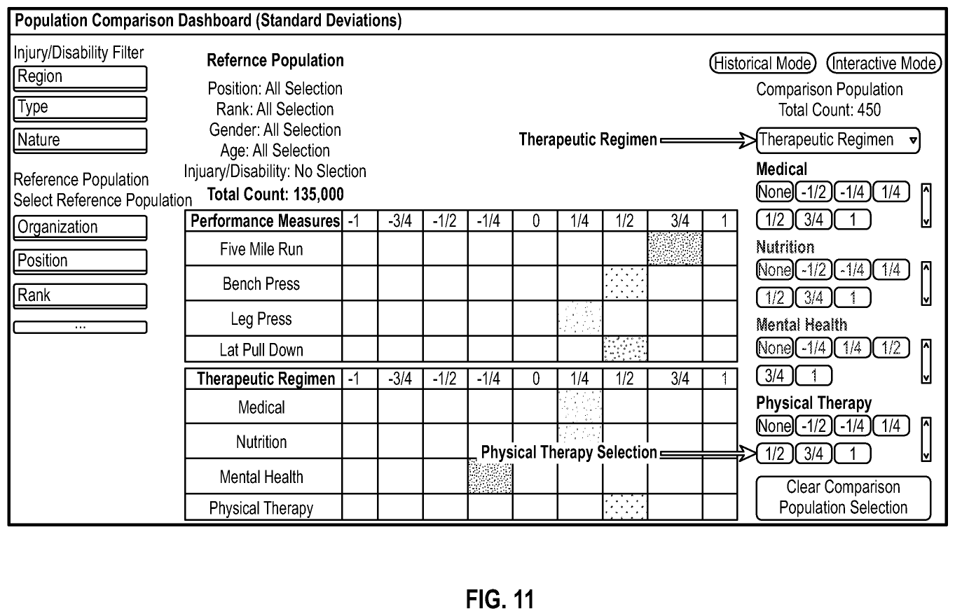

FIG. 11 shows an example user interface with interactive mode selected, cleared populations and impairment filters. “+½” is selected for physical therapy. Note that the performance measures all rise. This may imply the positive effect of physical therapy on overall performance. However, also note that the Comparison Population now has a count of 450 individuals. This means that, out of over 135,000 individuals, only 450 of them have observations under physical therapy that are at least a one-half standard deviation from the median.

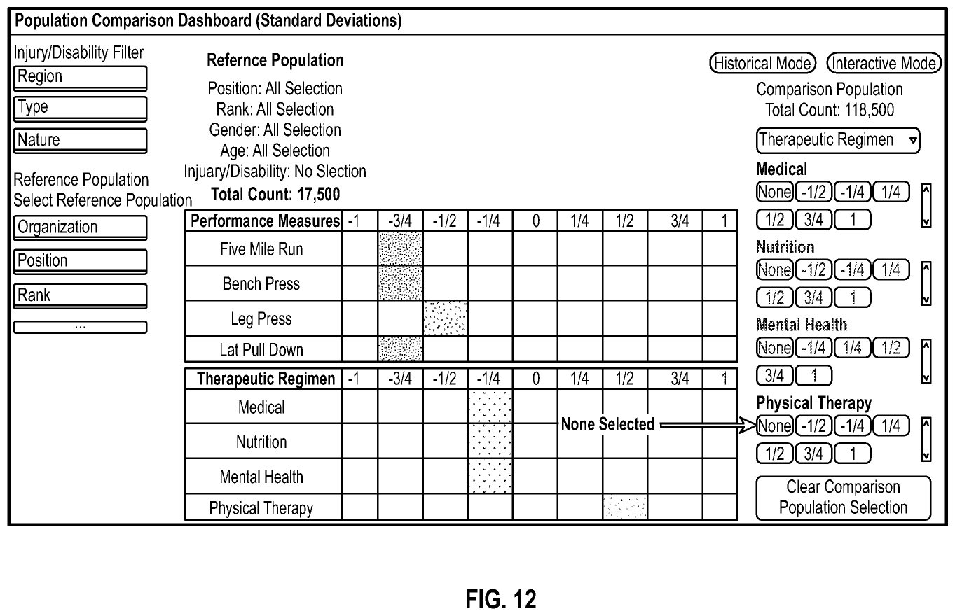

FIG. 12 shows an example user interface in interactive mode with the “None” option for the therapeutic regimen of “physical therapy.” Note that all metrics go negative. This allows a user to quickly look at the effect of having no therapeutic regimen. Selecting “none” here may imply that no therapeutic regimen results in worse performance overall, or conversely, may imply that physical therapy results in better performance overall in all performance measures. The continuous percentiles version works the same way, but, since the population is divided continuously into percentile buckets it may show better the amount of a change.

In the following discussion, a general description of the system and its components is provided, followed by a discussion of the operation of the same.

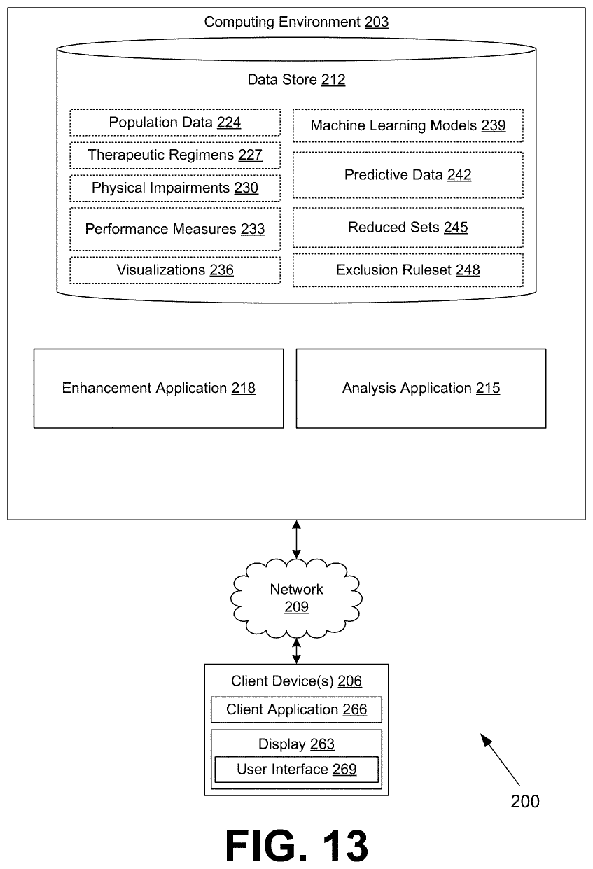

With reference to FIG. 13, shown is a networked environment 200 according to various embodiments. The networked environment 200 includes a computing environment 203 and one or more client devices 206, which are in data communication with each other via a network 209. The network 209 includes, for example, the Internet, intranets, extranets, wide area networks (WANs), local area networks (LANs), wired networks, wireless networks, cable networks, satellite networks, or other suitable networks, etc., or any combination of two or more such networks.

The computing environment 203 may comprise, for example, a server computer or any other system providing computing capability. Alternatively, the computing environment 203 may employ a plurality of computing devices that may be arranged, for example, in one or more server banks or computer banks or other arrangements. Such computing devices may be located in a single installation or may be distributed among many different geographical locations. For example, the computing environment 203 may include a plurality of computing devices that together may comprise a hosted computing resource, a grid computing resource, and/or any other distributed computing arrangement. In some cases, the computing environment 203 may correspond to an elastic computing resource where the allotted capacity of processing, network, storage, or other computing-related resources may vary over time.

Various applications and/or other functionality may be executed in the computing environment 203 according to various embodiments. Also, various data is stored in a data store 212 that is accessible to the computing environment 203. The data store 212 may be representative of a plurality of data stores 212 as can be appreciated. The data stored in the data store 212, for example, is associated with the operation of the various applications and/or functional entities described below.

The components executed on the computing environment 203, for example, include an analysis application 215, an enhancement application 218, and other applications, services, processes, systems, engines, or functionality not discussed in detail herein. The analysis application 215 is executed to perform analyses on population data 224 to determine insights with respect to the effect of therapeutic regimens on performance measures for individuals with physical impairments. The enhancement application 218 is executed to generate and update user interfaces that present the population data 224 to user in a historical or interactive view.

The data stored in the data store 212 includes, for example, population data 224, one or more therapeutic regimens 227, one or more physical impairments 230, one or more performance measures 233, one or more visualizations 236, one or more machine learning models 239, predictive data 242, one or more reduced sets 245, an exclusion ruleset 248, and potentially other data.

The population data 224 includes data describing or more populations of individuals who may have one or more physical impairments 230, which therapeutic regimens 227 are applied, and what the individuals' respective performance measures 233 are. The therapeutic regimens 227 may include various treatments that are imposed over time, like medical treatments, nutrition, physical therapy, conditioning regimens, psychiatric treatment, and so on. The therapeutic regimens 227 when applied to an individual in the population may be associated with a respective frequency (e.g., how often the therapeutic regimen is performed), a respective intensity (e.g., in what quantity, challenge level, or strength), and a respective duration (e.g., how long a time period).

The physical impairments 230 may include physical injuries or disabilities suffered by an individual, which may be associated with a corresponding region (e.g., knee, ankle, etc.), a corresponding type (e.g., contusion, ligament tear, fracture, etc.), and a corresponding nature (e.g., chronic, acute, etc.). It is noted that some or all individuals in a population may be impaired or may be unimpaired for comparison purposes.

The performance measures 233 are the results over time of various repeated physical activities like time for a five-mile run, amount of weight lifted in a bench press, and so on that represent an individual's state of physical fitness. The visualizations 236 correspond to tables, graphs, charts, and/or other graphical elements that can visually represent the impact of therapeutic regimens 227 on performance measures 233.

The machine learning models 239 are used by the analysis application 215 in order to perform various analyses on the population data 224. In particular, the machine learning models 239 may be trained on historical population data in order to generate predictive data 242, providing answers to various hypothetical scenarios selected by users. The machine learning models 239 may also generate reduced sets 245 of therapeutic regimens 227 and performance measures 233 to determine a fewer number of combinations that produce an optimal set of results. The exclusion ruleset 248 may be used in order to prevent the selection of variables that would result in an invalid or misleading presentation of the population data 224 or the predictive data 242.

The client device 206 is representative of a plurality of client devices that may be coupled to the network 209. The client device 206 may comprise, for example, a processor-based system such as a computer system. Such a computer system may be embodied in the form of a desktop computer, a laptop computer, personal digital assistants, cellular telephones, smartphones, set-top boxes, music players, web pads, tablet computer systems, game consoles, electronic book readers, smartwatches, head mounted displays, voice interface devices, or other devices. The client device 206 may include a display 263. The display 263 may comprise, for example, one or more devices such as liquid crystal display (LCD) displays, gas plasma-based flat panel displays, organic light emitting diode (OLED) displays, electrophoretic ink (E ink) displays, LCD projectors, or other types of display devices, etc.

The client device 206 may be configured to execute various applications such as a client application 266 and/or other applications. The client application 266 may be executed in a client device 206, for example, to access network content served up by the computing environment 203 and/or other servers, thereby rendering a user interface 269 on the display 263. To this end, the client application 266 may comprise, for example, a browser, a dedicated application, etc., and the user interface 269 may comprise a network page, an application screen, etc. The client device 206 may be configured to execute applications beyond the client application 266 such as, for example, email applications, social networking applications, word processors, spreadsheets, and/or other applications.

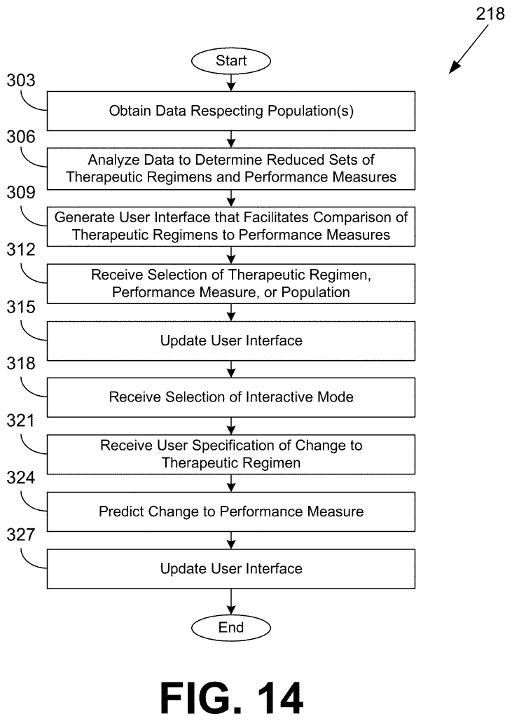

Referring next to FIG. 14, shown is a flowchart that provides one example of the operation of a portion of the enhancement application 218 according to various embodiments. It is understood that the flowchart of FIG. 14 provides merely an example of the many different types of functional arrangements that may be employed to implement the operation of the portion of the enhancement application 218 as described herein. As an alternative, the flowchart of FIG. 14 may be viewed as depicting an example of elements of a method implemented in the computing environment 203 (FIG. 13) according to one or more embodiments.

Beginning with box 303, the enhancement application 218 receives population data 224 respecting one or more populations of individuals, where the individuals may have one or more physical impairments 230. The population data 224 may describe one or more respective therapeutic regimens 227 that have been applied to the individuals, and the results of one or more performance measures 233, with the results potentially provided over time, before the physical impairments 230, before the therapeutic regimens 227 were started, during the therapeutic regimens 227, and after the therapeutic regimens 227. The population data 224 may be considered historical data, as the data corresponds to actual population members with the actual physical impairments 230, therapeutic regimens 227, and performance measures 233.

In box 306, the enhancement application 218 uses the analysis application 215 to analyze the population data 224 to determine reduced sets 245 of both the therapeutic regimens 227 and the performance measures 233. For example, there may be hundreds of different types of therapeutic regimens 227 and performance measures 223, but the analysis application 215 may apply a machine learning model 239 in order to determine an optimal combination of some number of therapeutic regimens 227 with some number of performance measures 233 to present useful results that are not overwhelming or cluttered by the user. For example, the machine learning model 239 may be trained on user feedback to determine which improvements are meaningful and also to exclude therapeutic regimens 227 that are not primary drivers of recovery.

In box 309, the enhancement application 218 generates a user interface 269 that facilitates a comparison of the reduced sets 245 of the therapeutic regimens 227 to the performance measures 233. The user interface 269 may include one or more visualizations 236 that enable a user to see the improvement or lack thereof over time as the therapeutic regimen 227 is applied. The user interface 269 may include components that facilitate selection and comparison of different populations, different types of physical impairments 230, and so forth.

In box 312, the enhancement application 218 receives a selection of a component associated with a therapeutic regimen 227, a performance measure 233, or a population. These components may enable manual inclusion or exclusion of the selected therapeutic regimen 227, performance measure 233, or population. In box 315, the enhancement application 218 updates the user interface 269 based upon the selections.

In box 318, the enhancement application 218 receives a selection of an interactive mode, which allows for hypothetical scenarios to be tested against the historical population data 224 using a machine learning model 239. In box 321, the enhancement application 218 receives a user specification of a change to a therapeutic regimen 227. In other examples, the change may be with respect to a physical impairment 230, or a population. In box 324, the enhancement application 218 uses the analysis application 215 to predict using a machine learning model 239 the change to the corresponding performance measures 233, thereby generating the predictive data 242.

In box 327, the enhancement application 218 updates the user interface 269 to show the predicted change. In some cases, the exclusion ruleset 248 may prevent the predicted change to be shown if the exclusion ruleset 248 causes the results to be excluded as being invalid or misleading. For example, the results may be based on insufficient data, or the resulting changes may be statistically insignificant. Thereafter, the operation of the portion of the enhancement application 218 ends.

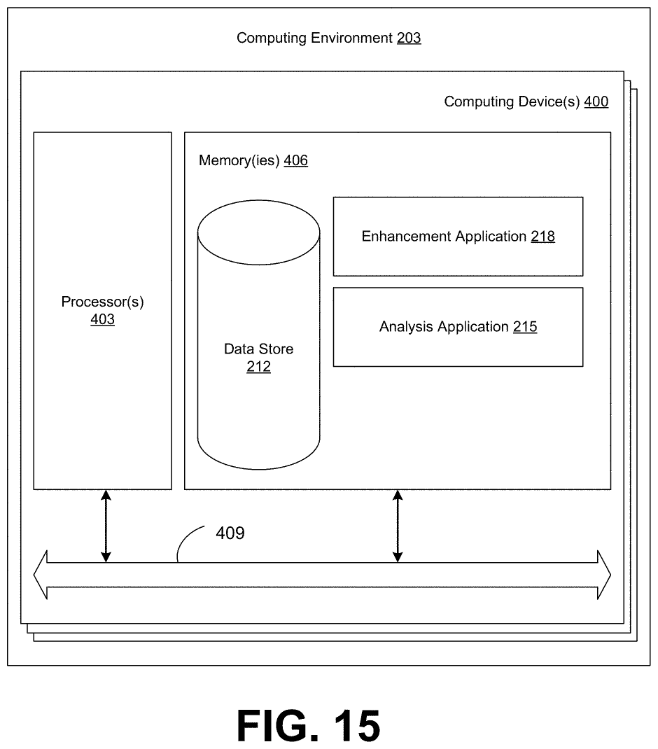

With reference to FIG. 15, shown is a schematic block diagram of the computing environment 203 according to an embodiment of the present disclosure. The computing environment 203 includes one or more computing devices 400 Each computing device 400 includes at least one processor circuit, for example, having a processor 403 and a memory 406, both of which are coupled to a local interface 409. To this end, each computing device 400 may comprise, for example, at least one server computer or like device. The local interface 409 may comprise, for example, a data bus with an accompanying address/control bus or other bus structure as can be appreciated.

Stored in the memory 406 are both data and several components that are executable by the processor 403. In particular, stored in the memory 406 and executable by the processor 403 are the analysis application 215, the enhancement application 218, and potentially other applications. Also stored in the memory 406 may be a data store 212 and other data. In addition, an operating system may be stored in the memory 406 and executable by the processor 403.

It is understood that there may be other applications that are stored in the memory 406 and are executable by the processor 403 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 406 and are executable by the processor 403. In this respect, the term “executable” means a program file that is in a form that can ultimately be run by the processor 403. Examples of executable programs may be, for example, a compiled program that can be translated into machine code in a format that can be loaded into a random access portion of the memory 406 and run by the processor 403, source code that may be expressed in proper format such as object code that is capable of being loaded into a random access portion of the memory 406 and executed by the processor 403, or source code that may be interpreted by another executable program to generate instructions in a random access portion of the memory 406 to be executed by the processor 403, etc. An executable program may be stored in any portion or component of the memory 406 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 406 is defined herein as including both volatile and nonvolatile memory and data storage components. Volatile components are those that do not retain data values upon loss of power. Nonvolatile components are those that retain data upon a loss of power. Thus, the memory 406 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 403 may represent multiple processors 403 and/or multiple processor cores and the memory 406 may represent multiple memories 406 that operate in parallel processing circuits, respectively. In such a case, the local interface 409 may be an appropriate network that facilitates communication between any two of the multiple processors 403, between any processor 403 and any of the memories 406, or between any two of the memories 406, etc. The local interface 409 may comprise additional systems designed to coordinate this communication, including, for example, performing load balancing. The processor 403 may be of electrical or of some other available construction.

Although the analysis application 215, the enhancement application 218, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowchart of FIG. 14 shows the functionality and operation of an implementation of portions of the enhancement application 218. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 403 in a computer system or other system. The machine code may be converted from the source code, etc. If embodied in hardware, each block may represent a circuit or a number of interconnected circuits to implement the specified logical function(s).

Although the flowchart of FIG. 14 shows a specific order of execution, it is understood that the order of execution may differ from that which is depicted. For example, the order of execution of two or more blocks may be scrambled relative to the order shown. Also, two or more blocks shown in succession in FIG. 14 may be executed concurrently or with partial concurrence. Further, in some embodiments, one or more of the blocks shown in FIG. 14 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the analysis application 215 and the enhancement application 218, that comprises software or code can be embodied in any non-transitory computer-readable medium for use by or in connection with an instruction execution system such as, for example, a processor 403 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the analysis application 215 and the enhancement application 218, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 400, or in multiple computing devices 400 in the same computing environment 203.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A non-transitory computer-readable medium embodying a program executable in at least one computing device, wherein when executed the program causes the at least one computing device to at least:
obtain data respecting a plurality of different populations of individuals having a respective physical impairment, the data indicating for each of the individuals a respective subset of a set of therapeutic regimens used to treat the respective physical impairment, and a set of performance measures of the respective individual following the respective subset of the set of therapeutic regimens;
analyze the data using a machine learning model to automatically determine a reduced set of therapeutic regimens and a reduced set of performance measures; and
generate a user interface that facilitates a comparison of individual ones of the reduced set of therapeutic regimens to the reduced set of performance measures for a selected subset of the plurality of different populations.

2. The non-transitory computer-readable medium of claim 1, wherein the user interface includes a selection component to select a particular physical impairment for analyzing the data and presenting the comparison, wherein the particular physical impairment is selected from a plurality of physical impairments.

3. The non-transitory computer-readable medium of claim 1, wherein when executed the program further causes the at least one computing device to at least:
receive a selection of an interactive mode via the user interface;
receive a user specification of a change to one or more of the reduced set of therapeutic regimens;
predict based at least in part on an analysis of the data a change to at least one of the performance measures from the reduced set of performance measures; and
update the user interface to display the change to the at least one of the performance measures.

4. A system, comprising:
at least one computing device; and
instructions executable in the at least one computing device, wherein when executed the instructions cause the at least one computing device to at least:
obtain data respecting at least one population of individuals having a respective physical impairment, the data indicating for each of the individuals a respective subset of a set of therapeutic regimens used to treat the respective physical impairment, and a set of performance measures of the respective individual following the respective subset of the set of therapeutic regimens;
analyze the data to automatically determine a reduced set of therapeutic regimens and a reduced set of performance measures; and
generate a user interface that facilitates a comparison of individual ones of the reduced set of therapeutic regimens to the reduced set of performance measures for the at least one population.

5. The system of claim 4, wherein the user interface includes a selection component to manually add or remove a therapeutic regimen from the reduced set of therapeutic regimens for the comparison.

6. The system of claim 4, wherein the user interface includes a selection component to manually add or remove a performance measure from the reduced set of performance measures for the comparison.

7. The system of claim 4, wherein the at least one population comprises a plurality of populations of individuals, and the user interface includes a selection component to select a subset of the plurality of populations of individuals.

8. The system of claim 4, wherein analyzing the data to automatically determine the reduced set of therapeutic regimens and the reduced set of performance measures uses a machine learning model.

9. The system of claim 4, wherein when executed the instructions further cause the at least one computing device to at least:
receive a selection of an interactive mode via the user interface;
receive a user specification of a change to one or more of the reduced set of therapeutic regimens;
predict based at least in part on an analysis of the data a change to at least one of the performance measures from the reduced set of performance measures; and
update the user interface to display the change to the at least one of the performance measures.

10. A method, comprising:
obtaining, by at least one computing device, data respecting at least one population of individuals having a respective physical impairment, the data indicating for each of the individuals a respective subset of a set of therapeutic regimens used to treat the respective physical impairment, and a set of performance measures of the respective individual following the respective subset of the set of therapeutic regimens;
analyzing, by the at least one computing device, the data to automatically determine a reduced set of therapeutic regimens and a reduced set of performance measures; and
generating, by the at least one computing device, a user interface that facilitates a comparison of individual ones of the reduced set of therapeutic regimens to the reduced set of performance measures for the at least one population.

11. The method of claim 10, wherein the user interface includes a selection component to manually add or remove a therapeutic regimen from the reduced set of therapeutic regimens for the comparison.

12. The method of claim 10, wherein the user interface includes a selection component to manually add or remove a performance measure from the reduced set of performance measures for the comparison.

13. The method of claim 10, wherein the user interface includes a selection component to filter the respective physical impairment by a corresponding region, a corresponding type, and a corresponding nature.

14. The method of claim 10, wherein one or more of the reduced set of therapeutic regimens are associated with a respective frequency, a respective intensity, and a respective duration.

15. The method of claim 10, wherein the user interface includes a selection component to select a particular physical impairment for analyzing the data and presenting the comparison, wherein the particular physical impairment is selected from a plurality of physical impairments.

16. The method of claim 10, wherein the at least one population comprises a plurality of populations of individuals, and the user interface includes a selection component to select a subset of the plurality of populations of individuals.

17. The method of claim 10, wherein analyzing the data to automatically determine the reduced set of therapeutic regimens and the reduced set of performance measures uses a machine learning model.

18. The method of claim 10, further comprising:
receiving, by the at least one computing device, a selection of an interactive mode via the user interface;
receiving, by the at least one computing device, a user specification of a change to one or more of the reduced set of therapeutic regimens;
predicting, by the at least one computing device, based at least in part on an analysis of the data a change to at least one of the performance measures from the reduced set of performance measures; and
updating, by the at least one computing device, the user interface to display the change to the at least one of the performance measures.

19. The method of claim 18, wherein the change to the one or more of the reduced set of therapeutic regimens corresponds to a maximization of the one or more of the reduced set of therapeutic regimens.

20. The method of claim 18, further comprising verifying, by the at least one computing device, that the change to the one or more of the reduced set of therapeutic regimens would not produce a prediction that is invalid or misleading before updating the user interface.
